(19) United States
(12) Patent Application Publication
(10) Pub. No.: US 2021/0330849 A1
(43) Pub. Date: Oct. 28, 2021

(54) SANITIZATION INDICATOR

(71) Applicant: JEANOLOGIA TEKNOLOJI A.S., Izmir (TR)

(72) Inventors: Merih ÖZÇELIK, Izmir (TR); Hüseyin AYDIN, Izmir (TR)

(21) Appl. No.: 16/869,011

(22) Filed: May 7, 2020

(30) Foreign Application Priority Data
Apr. 28, 2020 (TR) 2020/06637

(51) Int. Cl. A61L 2/28; B08B 5/00; A61L 2/20

(52) U.S. Cl. CPC A61L 2/28; B08B 5/00; A61L 2202/26; B08B 2205/00; A61L 2202/14; A61L 2/202

(57) ABSTRACT

Disclosed is a sanitization indicator that indicates whether or not sanitization has been performed after textile products such as sweaters, t-shirts, trousers, skirts, shirts, shoes, which are sold in stores, are worn and removed in fitting rooms.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

### 1. Field of the Invention

The invention relates to a sanitization indicator that indicates whether or not sanitization is performed after textile products such as sweaters, t-shirts, trousers, skirts, shirts, shoes, which are sold in stores, are worn and removed in the test or fitting rooms.

### 2. Description of Related Art Including Information Disclosed Under 37 CFR 1.97 and 37 CFR 1.98

Sanitization is generally the procedure required for cleaning and disinfection on any surface. Disinfection, which includes the reduction of microorganisms present in the environment and the elimination of all pathogenic microbes, has been a factor directly affecting human health in today's world.

Microorganisms such as viruses and bacteria can be transmitted directly from person to person as well as transmitted from person to person on an object. Foodstuffs, daily goods and most importantly, the textile products we use as garments contain the most suitable environment for the mentioned microorganisms to pass to human beings.

Although clothes are structures suitable for personal use of people, it is necessary to try them in fitting rooms in order to determine whether the clothes mentioned in the stores where the clothes are sold are suitable for our body or not. After these trials, if the product has not been purchased, the said textile products are placed on the store shelves by the shop assistant, ready for the next customer to try on.

This method, which is applied in all stores in the current technique, causes a faster spread of diseases caused by viruses and bacteria in today's world.

The user who tries on the textile product, virus or bacteria that spread around due to the particles that they have during the trial or come out of the mouth by coughing or sneezing, are smeared on the textile product. This contamination causes contamination of the user who tries the next textile product, even the shop assistant who takes the textile product back to the old place in the store.

For these reasons, the mentioned textile products should be subjected to sanitization after each trial. Only in this way can textile products that are put on their shelves after this process become safer for the second user.

Although the sanitization process mentioned in today's technology can be done in stores, this is a process that requires constant control and it is not possible for users to confirm.

In other words, the user does not know whether the textile product he will try has been tried on before him or not, and if it has been tried on, it is disinfected in accordance with the reuse. In such a situation, the store has to trust the information given by the officers or gives up to try the textile product because it does not.

Since there is no confirmation process, the store staff does or does not do this on his own initiative. There is no system that controls this.

In the literature the Korean patent application numbered KR101875370 relates to “The present invention relates to a carrier with a clothing holding part generating ion, comprising: a carrier body having a structure capable of being opened/closed to store articles in the inside; the clothing holding part; and an anion generator installed in a storage space to provide anion. For the clothing holding part: a detachable cover is detachably installed on one side surface of the carrier body; a holding space is formed inside the carrier body to be opened by separation of the detachable cover; the detachable cover is fixed to be placed on an upper side of the holding space by a support detachably installed in the carrier body; a hanging bar is formed on a side facing the storage space from the detachable cover such that a hanger is hung on the hanging bar; and a top and a bottom are connected to the edge boundary of the detachable cover and the edge boundary of an entrance side of the holding space to be able to be separated, respectively, such that the clothing holding part upwardly expands the storage space, which is isolated to receive clothing hung on the hanger, from the holding space. According to the present invention, a space for holding the clothing is provided in a carrier, thereby conveniently storing the clothing through winkle prevention of the clothing or the like. Moreover, anion is generated in the storage space of the clothing, thereby allowing a user to pleasantly wear the clothing through sterilization, deodorization and the like of the clothing.”.

In the aforementioned application, a device with a portable structure that sterilizes the clothes is disclosed.

In the literature the China patent application numbered CN110012568 relates to “The invention provides a textile fabric with a heating function, which comprises a heating layer and a conductive layer, wherein the heating layer is connected to the upper end face of the conductivelayer, the heating layer includes silver-plated conductive fibers, nickel-chromium filaments and polyester modified fibers, and the conductive layer includes copper wires, cotton fibers, tencel fibers and nylon fibers. Compared with the prior art, the textile fabric has the beneficial effects that the nickel-chromium filaments in the heating layer and the copper wires in the conductive layer form an electrifying circuit, the automatic heating of the nickel-chromium filaments is realized, and thus the heat preservation effect is improved. In addition, it is convenient to store the heat in the case of power interruption through the polymer modified fibers, and the heat preservation time is prolonged. The textile fabric has the functions of sterilization and anti-ultraviolet radiation, and has the advantages of soft hand feeling, fluffiness, moisture absorption, quick drying and portability.”

In the said application, a portable device used for sterilization of textile products is disclosed.

In the literature the China patent application numbered CN103882685 relates to “The invention discloses a disinfection treatment method for a medical non-woven fabric. The non-woven fabric is prepared through successive antistatic treatment, plasma treatment and disinfection treatment. In the step of antistatic treatment, the non-woven fabric is put in an antistatic agent for soaking for 30 min and is then taken out for drying, wherein the antistatic agent contains phosphoric acid and sylvite. Plasma treatment refers to oxidizing gas plasma aeration treatment. In the step of disinfection treatment, the non-woven fabric is soaked in ethanol with a concentration of more than 95% for washing twice to three times and is then hermetically packaged. According to the invention, adhesion of a plurality of fabrics is avoided through antistatic treatment; the medical non-woven fabric is hermetically packaged, so the fabric is convenient to store and carry and has a long period of validity; since the fabric is washed with ethanol with a concentration of more than 95% twice to three times, effective sterilization and water impermeability are ensured; and the medical non-woven fabric has an obvious pain relieving effect and is helpful for wound healing.”

In the said application, it describes a device and method that is disinfected through chemical processes.

Although it is possible to carry out sanitization of textile products with different methods, there is no system that controls and confirms them.

Due to the above-mentioned disadvantages there was a need to introduce a sanitization indicator.

### BRIEF SUMMARY OF THE INVENTION

Based on this position of the technique, the object of the invention is to provide a sanitization indicator that eliminates the existing disadvantages.

Another object of the invention is to provide a structure that allows sanitization to be performed before the products tested in the fitting rooms in the stores are tried by a second user.

Another object of the invention is to provide a structure that prevents users from being exposed to microorganisms such as viruses and bacteria from textile products.

Another object of the invention is to provide a structure that enables textile stores to work in a much safer environment.

Another object of the invention is to provide a structure that increases the trust of the users in the stores selling textile products.

Another object of the invention is to provide a structure in which it can be checked whether the store staff performs sanitization.

### BRIEF DESCRIPTION OF THE SEVERAL VIEWS OF THE DRAWINGS

FIG. 1 is representative view of the sanitization indicator subject to the invention before sanitization.

FIG. 2 is a representative view of the textile product subject to the invention showing sanitization in the ozonation unit.

FIG. 3 is a representative view of the sanitization indicator subject to the invention after the sanitization process.

FIG. 4 is a representative view of the sanitization indicator subject to the invention on the textile product after the sanitization process.

### REFERENCE NUMBERS

1. Textile Product
1.1 Waist/Neck Portion
2. Sanitization Indicator
2.1 Confirmation Letter
2.2 Security Connection Slot
3. Ozonation Unit
4. Security Connection Apparatus

### DETAILED DESCRIPTION OF THE INVENTION

In this detailed explanation, the innovation of the invention is explained only with examples that will have no limiting effect on better understanding of the subject.

The invention is a sanitization confirmation system comprising an ozonation unit (3) located in said stores to enable the sanitization process after the removal of textile products (1) which put up for sale in the stores such as sweaters, t-shirts, trousers, skirts, shirts, shoes after being worn and removed from the test rooms characterized in that it comprise a sanitization indicator (2) positioned on said textile product (1) indicating whether said textile products (1) have been sanitized in the ozonation unit (3) and confirmation letter (2.1) written on said sanitization indicator (2), which occurs only after reaction with ozone and color change and written with a chemical that changes color in reaction with ozone.

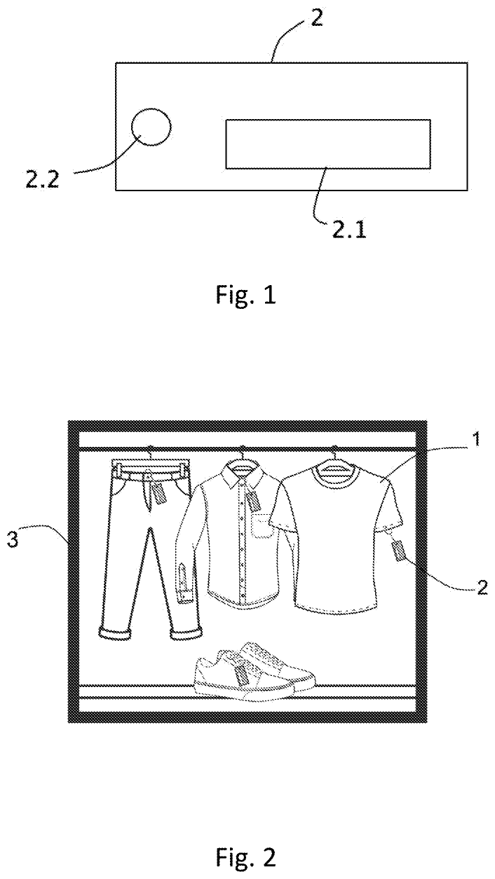

FIG. 1 shows a representative view of the sanitization indicator (2), which is the subject of the invention, is shown before sanitization.

FIG. 2 shows a representative view of the textile product (1), which is the subject of the invention, is shown inside the ozonation unit (3).

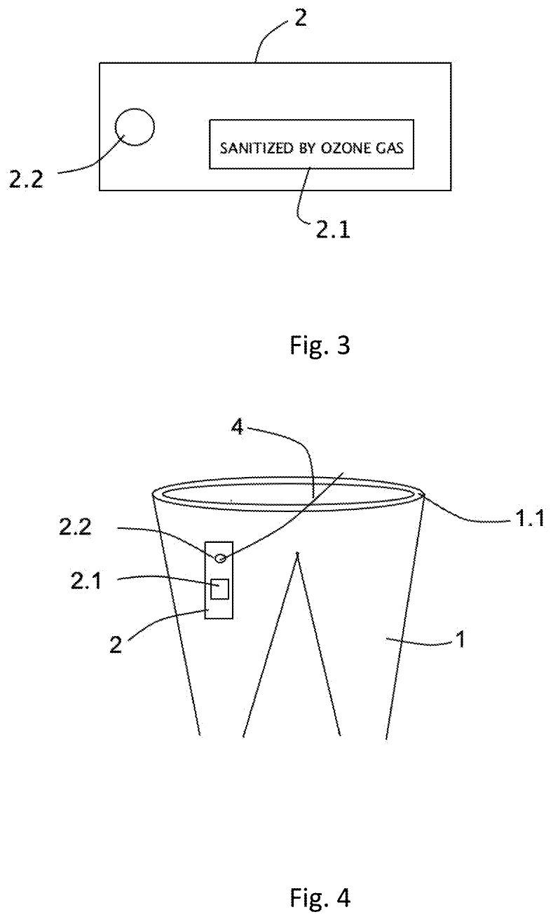

FIG. 3 shows a representative view of the sanitization indicator (2) which is the subject of the invention is shown after the sanitization process.

FIG. 4 shows a representative view of the sanitization indicator (2) which is the subject of the invention on the textile product (1) after the sanitization process is illustrated.

After the textile products (1) offered for sale in the stores are tested by a user in the fitting rooms, the store personnel first fixes the sanitization indicator (2) on the textile product (1) to be subjected to sanitization and then it is located in the ozonation unit (3) located in the store.

Before the said sanitization indicator (2) enters the ozonation unit (3), it has an invisible confirmation letter (2.1). Said confirmation letter (2.1) is written with a chemical that oxidizes and changes color by reacting with ozone.

The confirmation text (2.1) mentioned in a preferred embodiment of the invention is a chemical, potassium iodide, or another chemical that oxidizes and changes color in its reaction with ozone.

The sanitization indicator (2) in which said confirmation letter (2.1) is located, is fixed on a security connection apparatus (4) so as to pass both sides of said textile product (1), preferably at the waist/neck portion (1.1).

In the preferred embodiment of the present invention, said safety connection apparatus (4) is a material that will bring two sides together at the waist/neck portion (1.1) such as fishbone, rope and the like, and also carry said sanitization indicator (2).

The security connection slot (2.2) formed on said sanitization indicator (2) provides its connection with the textile product (1) by passing through said security connection apparatus (4).

When the store personnel operates said ozonation unit (3), it provides sterilization of the ozone textile product (1) in said ozonation unit (3), at the same time, the confirmation letter (2.1) on the sanitization indicator (2) fixed on said textile product (1) is oxidized. and thus enables color change.

Thus, with the appearance of the confirmation letter (2.1), the sanitization indicator (2) indicates to the user that the said textile product (1) has been sanitized. Thus, the user can test the textile product much more safely.

When another user wants to try the textile product (1) which has undergone sanitization, he has to break the security connection apparatus (4) that fixes both sides of the textile product (1) at the waist/neck portion (1.1). Otherwise, he cannot wear the textile product (1).

Thus, the sanitization indicator (2) appearing in the said confirmation letter (2.1) is separated from the said textile product (1) and shows that it is not sanitized and unsafe before attempting to the next user.

In order to ensure this security and to allow the next user to try on the textile product (1) with peace of mind, the store personnel must fix it again by fixing a new sanitization indicator (2) to the ozonation unit (3) and ensure that the confirmation letter (2.1) appears on the new sanitization indicator (2).

Said confirmation letter (2.1) contains the words that indicate that said textile product (1) is subjected to sanitization.

We claim:

1. A sanitization confirmation system comprising an ozonation unit located in stores to enable the sanitization process after the removal of textile products which are put up for sale in the stores such as sweaters, t-shirts, trousers, skirts, shirts, shoes after being worn and removed from fitting rooms, characterized in that the system comprises a sanitization indicator positioned on said textile product indicating whether said textile products have been sanitized in the ozonation unit and a confirmation letter written on said sanitization indicator, which occurs only after reaction with ozone and color change and written with a chemical that changes color in reaction with ozone.

2. A confirmation letter according to claim 1, wherein said chemical contains potassium iodide.

3. Sanitization confirmation system according to claim 1, comprising a security connection apparatus that fixes the waist/neck portion of said textile product from both sides and carries the sanitization indicator on said textile product.

4. Sanitization confirmation system according to claim 1, said sanitization indicator comprising a security connection slot that supports the security connection apparatus to provide connection with said textile product.

5. Sanitization confirmation system according to claim 1, said security connection apparatus is in the structure to bring together the two sides at the waist/neck part such as fishbone, rope and the like, and also to carry said sanitization indicator.

6. Sanitization confirmation system according to claim 1, wherein said confirmation letter contains words indicating that said textile product has been subjected to sanitization.
